(19) United States
(12) Patent Application Publication
(10) Pub. No.: US 2019/0314169 A1
(43) Pub. Date: Oct. 17, 2019

(54) COATED AND LAYERED VERTEBRAL DISC ARTHRODESIS PROTHESIS

(71) Applicants: Amit Patel, Timonium, MD (US); Manoj K. Mishra, Woodstock, MD (US); Deven Patel, Cedar Grove, NJ (US)

(72) Inventors: Amit Patel, Timonium, MD (US); Manoj K. Mishra, Woodstock, MD (US); Deven Patel, Cedar Grove, NJ (US)

(21) Appl. No.: 16/384,686

(22) Filed: Apr. 15, 2019

Related U.S. Application Data
(60) Provisional application No. 62/658,557, filed on Apr. 16, 2018.

(51) Int. Cl. A61F 2/44; A61F 2/30

(52) U.S. Cl. CPC A61F 2/4455; A61F 2/30767; A61F 2002/3092; A61F 2002/4475; A61F 2002/3093; A61F 2002/30014

(57) ABSTRACT

A vertebral disc including a superior surface layer, an inferior surface layer, a midsection between the superior and inferior surface layers and a hollow center. The superior surface layer and inferior surface layer each comprise a layer of lattice with pore sizes in the range of 200-900 μm and a porosity of each layer of 55-75%. The midsection of the implant comprises a random or pseudorandom lattice interposed between the superior surface layer and inferior surface layer with larger, macro-pores of various sizes. There is a gradient of pore sizes along a longitudinal axis with the smallest pore sizes comprising the superior and inferior surfaces and the largest pore sizes comprising the midsection.

### CROSS REFERENCE TO RELATED APPLICATION

The subject application claims the benefit of and priority to Provisional Patent Application No. 62/658,557, filed on Apr. 16, 2018, the disclosure of which is herein incorporated by reference in its entirety.

### FIELD OF THE INVENTION

This invention pertains to a prosthetic insert for spinal arthrodesis.

### BACKGROUND

Spinal pathologies and disorders such as scoliosis and other curvature abnormalities, kyphosis, degenerative disc disease, disc herniation, osteoporosis, spondylolisthesis, stenosis, tumor, and fractures may result from factors including trauma, disease, and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction discectomy, laminectomy and implantable prosthetics. As part of these surgical treatments, spinal constructs for arthrodesis, such as, for example, bone fasteners, spinal rods and interbody devices can be used to provide stability to a treated region. For example, during surgical treatment, interbody implants can be delivered to a surgical site for fixation with bone to immobilize a joint. In another embodiment in spinal treatments, deteriorated spinal discs can be replaces with an implant to provide the natural spacing between vertebrae and to mimic, as closely as possible, the flexibility of natural cartilage discs. For example, deteriorated spinal discs include herniated discs.

An approach to a spinal implant is disclosed in PCT International Patent Application WO2017/106780, which described a layered device having irregular pores. Another approach is described in U.S. Pat. No. 9,662,226, which described an implant having a hollow boxy structure with regularly spaced circular openings.

### SUMMARY OF THE INVENTION

A vertebral disc including a superior surface layer, an inferior surface layer, a midsection between the superior and inferior surface layers and a hollow center. The superior surface layer and inferior surface layer each comprise a layer of lattice with pore sizes in the range of 200-900 μm and a porosity of each layer of 55-75%. The midsection of the implant comprises a random or pseudorandom lattice interposed between the superior surface layer and inferior surface layer with larger, macro-pores of various sizes. There is a gradient of pore sizes along a longitudinal axis with the smallest pore sizes comprising the superior and inferior surfaces and the largest pore sizes comprising the midsection.

The lattices of superior surface layer and inferior surface layer each include a tantalum alloy or titanium alloy material of 0.5 mm to 2 mm thickness. The lattice of the midsection includes a tantalum alloy or titanium alloy material with a thickness of between 4 mm and 14 mm. The lattices of the superior surface layer, the inferior surface layer and the midsection are roughened with a macro surface roughness. The lattices of the superior surface layer, inferior surface layer and the midsection are coated with hydroxyapatite (HA) or tricalcium phosphate (TCP) with a coating thickness of approximately 25-35 microns (μm). The transition of pore sizes from the superior and inferior surface layers to the midsection is gradual.

The superior surface layer and inferior surface layer each contact a vertebra. In an embodiment the superior surface layer and inferior surface layer each define a plane such that the superior surface plane and inferior surface plane are parallel. In an additional embodiment, the superior surface layer and inferior surface layer each define a plane and the superior surface plane and the inferior surface plane are non-parallel and at a 5° to 30° angle with respect to each other. More specifically, a height at a first end of the midsection is less than a height of a second opposing end of the midsection.

A vertebral disc prosthesis for cervical, thoracic, lumbar disc arthrodesis including a superior surface layer, an inferior surface layer, a midsection, and a hollow center on a longitudinal axis to serve as a graft window. The superior surface layer and inferior surface layer each include an approximately 0.5 mm to 2 mm thick layer of lattice made from tantalum alloy or titanium alloy material. The superior and inferior surface layers further include pore sizes in the spongy matrix in the range of 200-900 μm with a porosity of each layer of 55-75%. The midsection of the implant includes a random or pseudorandom lattice interposed between the superior surface layer and inferior surface layer. The lattice of the midsection includes tantalum alloy or titanium alloy material with a thickness of between 4 mm and 14 mm and a pore size of 0.5 mm to 5.0 mm. There is a gradient of pore sizes along the longitudinal axis with the smallest pore sizes comprising the superior and inferior surfaces and the largest pore sizes comprising the midsection. A vertebral disc prosthesis for cervical, thoracic, lumbar disc arthrodesis including a circular (torodial), kidney, or parallelepipedal shaped implant. The implant has a superior surface layer, an inferior surface layer, a midsection, and a hollow center on a longitudinal axis to serve as a graft window. Each surface layer contacts a vertebra. The superior surface layer and inferior surface layer each includes an approximately 0.5 mm to 2 mm thick layer of lattice made from tantalum alloy or titanium alloy material and pore sizes in the spongy matrix in the range of 200-900 μm, with a porosity of each layer of 55-75%. The midsection of the implant comprises a random or pseudorandom lattice interposed between the superior surface layer and inferior surface layer. The lattice of the midsection comprises tantalum alloy or titanium alloy material with a thickness of between 4 mm and 14 mm and a pore size of 0.5 mm to 5.0 mm. The lattices of the superior surface layer, inferior surface layer and midsection are roughened with a macro surface roughness. In addition, the lattices of the superior surface layer, inferior surface layer and midsection are coated with hydroxyapatite (HA) or tricalcium phosphate (TCP), with a coating thickness of approximately 25-35 microns (um).

The features described above and herein of the implant of the subject invention and the manner in which it is manufactured and employed will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the subject invention relates will readily understand how to make and use the implant of the subject invention without undue experimentation, embodiments thereof will be described in detail herein below with reference to certain figures, wherein:

FIG. 1 is a perspective view of an embodiment of the vertebral disc prosthesis for cervical, thoracic, lumbar disc arthrodesis, with parallel superior and inferior layers.

FIG. 2 is a side elevation of an embodiment of the vertebral disc prosthesis for cervical, thoracic, lumbar disc arthrodesis, with parallel superior and inferior layers. The viewpoint is along the latitudinal axis.

FIG. 3 is a transparent view of an embodiment of the vertebral disc prosthesis for cervical, thoracic, lumbar disc arthrodesis.

FIG. 4 is a transparent view of an embodiment of the vertebral disc prosthesis for cervical, thoracic, lumbar disc, arthrodesis with the plane of the superior and inferior layers as a non-parallel angle.

FIG. 5 is a cross section of the L4 and L5 vertebrae showing the vertebral disc prosthesis for lumbar disc arthrodesis implanted between the L4 and L5 vertebrae.

### DETAILED DESCRIPTION

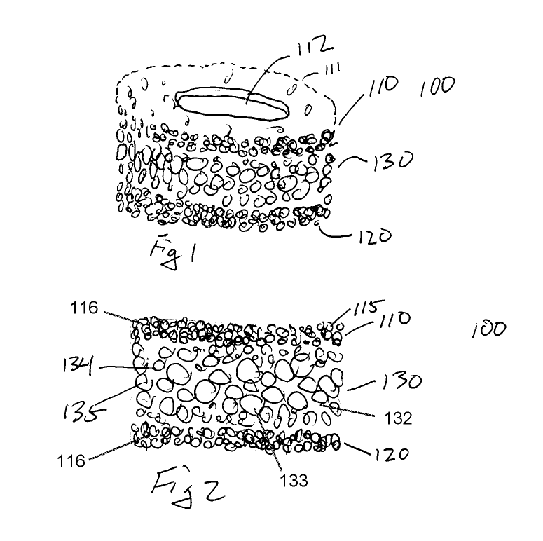

Referring now to the drawings, wherein like reference numerals identify similar structural features or aspects of the subject invention, there is illustrated in FIG. 1 an implant designated generally by reference numeral 100. The implant 100 is particularly well adapted for use in performing minimally invasive surgical procedures for treating damage of the intervertebral discs and/or the vertebral members.

It is envisioned however, that the implant 100 of the subject invention can be used in other spinal procedures as well, including, but not limited to as an adjunct to spinal fusion procedures, or as a spinal stabilization device. Those skilled in the art will readily appreciate from the following description that the implant 100 of the subject invention is well adapted for percutaneous insertion. That is, the implant 100 is dimensioned and configured for introduction and placement both through a small lateral skin incision as well as other insertion methods that may provide the least amount of additional trauma and nerve damage for the patient.

FIG. 1 shows an embodiment of the vertebral disc prosthesis 100 for cervical thoracic, lumbar disc arthrodesis of this invention. The prosthesis or implant 100 may have a circular (torodial), kidney, or parallelepipedal shape when viewed from above. FIG. 1 illustrates a perspective of a toroidal embodiment. The implant includes a superior surface layer 110 (i.e. top), an inferior surface layer 120 (i.e. bottom), a midsection 130 interposed between the superior and inferior layers, and a hollow center 112 on the longitudinal axis to serve as a graft window. In an embodiment, each of the surface layers forms a generally planar structure. The implant 100 has a longitudinal axis and a latitudinal axis. In an embodiment, at least one of the superior or inferior surface layers 110, 120 is perpendicular to the longitudinal axis.

In an embodiment, the implant is constructed from a suitable metal or metal alloy material, that can be fabricated into a solid from with a random or pseudorandom pattern of pores, constructed such that the pore size increases in the midsection as compared to the superior and inferior layer sections. In an embodiment, there is a gradient of pore sizes along the longitudinal axis with the smallest ports at the superior and inferior surfaces and the largest ports in the midsection.

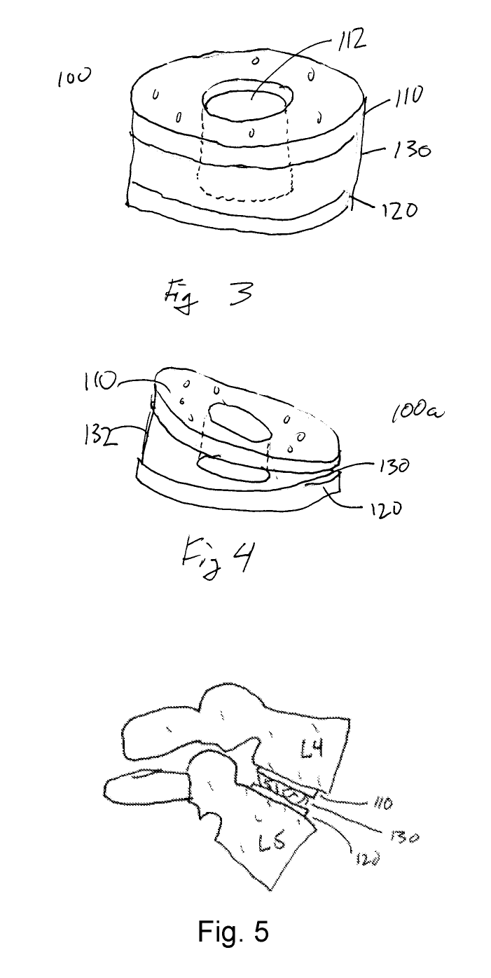

In an embodiment, each surface layer contacts a vertebra as shown in FIG. 5. In FIG. 5, the superior surface layer 110 is shown in contact with the L4 vertebra, and the inferior surface layer 120 is in contact with the L5 vertebra. FIG. 5 also shows the midsection 130 interposed between the two layers 110, 120.

FIG. 2 is an elevation view showing the prosthesis 100 viewed from the side. The superior surface layer 110 and the inferior surface layer 120 each comprise an approximately 2 mm thick layer of a lattice 116 made from tantalum alloy or titanium alloy material 115 of 0.05 mm to 0.25 mm thickness. In an embodiment, lattice 116 may be a spongy matrix, i.e., having an appearance and three-dimensional structure similar to a sponge. In an embodiment, the lattice 116 may have a similar texture to steel wool. The spongy matrix 116 may have pore sizes in the range of 200-900 μm, with a porosity of each layer of 55-75%. The porosity percentage is an expression of the ratio of space i.e., pores in the lattice, to the alloy material in the matrix. For example, with a 60% porosity, 60% of the volume of a given three-dimensional portion of the matrix would be air, and 40% of the space would be occupied by the alloy material in the lattice. A 60-65% porosity matches cancellous bone and is inductive to bone-in growth.

The midsection region 130 of the implant 100 comprises a lattice 132 interposed between the superior surface layer 110 and inferior surface layer 120. In an embodiment, the lattice 132 comprises a random or pseudorandom pattern of pores 133 wherein the solid material may be tantalum alloy or titanium alloy material 134 with thickness of between 4 mm and 14 mm, and a pore size of 0.5 mm to 5.0 mm.

The embodiment in FIG. 2 also illustrates that the boundaries between the superior layer 110, midsection 130 and inferior layer 120 may not be clearly demarcated. Rather, the transition from the alloy material of 0.05 mm to 0.25 mm thickness (115) and pore sizes in the range of 200-900 μm in the superior and inferior layers 110, 120 to the midsection 130 having struts with a thickness of between 0.5 mm and 2.5 mm (134) and a pore size of 0.5 mm to 5.0 mm (133) may be gradual. In an embodiment, there may be a smooth gradient of material thickness and pore size from the superior and inferior layers 110, 120 to the midsection 130, illustrated by 135 in FIG. 2.

The smooth gradient embodiment of the foregoing paragraph can be accomplished using additive manufacturing, which may also be termed 3D printing, in which the material is built up under computer control. Additive manufacturing permits significant flexibility in design and construction of the vertebral disc prosthesis of this invention. With additive manufacturing, intricate patterns of struts with varying patterns, material thickness, and pore sizes can be easily manufactured to provide a prosthesis that is essentially unitary, meaning it is essentially a single part that has not been assembled from a plurality of distinct parts.

In an embodiment, the pore size of the implant is larger in the latitudinal center, than at each latitudinal edge, i.e., the superior surface layer and inferior surface layer.

An alternative view of the vertebral disc prosthesis of this invention is shown in FIG. 3, as a transparent view. FIG. 3 is a schematic showing the overall structure and approximate boundaries of the superior layer 110, the inferior layer 120, the midsection 130 and hollow center 112.

In an embodiment, the superior surface layer and inferior surface layers 110, 120 each define a plane which are parallel, as shown in FIG. 3.

In an embodiment shown in FIG. 4, the planes defined by the superior surface layer 110 and inferior surface layers 120 are not parallel, and define an approximately 5° to 30° angle, such that the midsection is thinner at one edge of the vertebral disc prosthesis. More specifically, a height at a first end of the midsection 130 is less than a height at a second, opposing end of the midsection 130. This embodiment may be useful as a wedge-shaped prosthesis that comports the relationship of the vertebra in the natural curves of the spine. For example, when viewed from the side, a healthy spine defines an “S” shape and many of the natural intervertebral discs have a wedge configuration, that may be known as the lordotic wedge angle. For example, the L4-L5 wedge angle is normally 12°-22°. The L5-S1 wedge in normal spines is 16°-24° (http://sittingsafely.com/intervertebral-disc-angles/).

In an embodiment, the overall height of the implant 100 from an edge of the superior surface layer 110 to an edge of the inferior surface layer 120, ranges from 5 mm to 16 mm.

A particular advantage to the larger pores in the lattice of the midsection 130 is to reduce the amount of metal in the implant 100. The primary advantage of minimizing the amount of metal is easier imaging, especially with MRI and CT scan methods. Less metal in the implant will reduce the amount of scatter in imaging studies. In addition, the lattice work may have significant inherent flexibility imitating the natural flexibility of cartilage in natural intervertebral discs. Furthermore, the design is such to modulate the stiffness of the implant and better match the modulus of elasticity to that of native human bone.

In an embodiment, the entire implant 100 is fabricated from medically compatible tantalum, titanium, tantalum alloy or titanium alloy. For example, an appropriate titanium alloy may be titanium 6AL4V and 6AL4V ELI (ASTM Standard F1472, https://www.astm.org/Standards/F1472.htm; see also https://en.wikipedia.org/wiki/Ti-6Al-4V), which are alloys made with about 6% aluminum and 4% vanadium. An appropriate tantalum alloy may be tantalum alloyed with 2.5% to 10% tungsten, or 40% niobium. These materials are well known to have good biocompatibility.

Ideally, a bone implant or prosthesis would have a similar elastic modulus, also termed Youngs modulus, to natural bone. The modulus of elasticity for natural vertebra, which are cancellous bone, varies from about 0.1 GPa to 1.0 GPa (Biomaterials by freeze casting, Ulrike G. K. Wegst, Matthew Schecter, Amalie E. Donius, Phillip M. Hunger, Phil. Trans. R. Soc. A 2010 368 2099-2121; DOI 10.1098/rsta.2010.0014. Published 22 Mar. 2010; F. El Masri, E. Sapin de Brosses, K. Rhissassi, W. Skalli & D. Mitton (2011) Apparent Young's Modulus of vertebral cortico-cancellous bone specimens, Computer Methods in Biomechanics and Biomedical Engineering, 15:1, 23-28, DOI: 10.1080/10255842.2011.565751; http://en.wikipedia.org/wiki/Mechanical_properties_of_biomaterials). The more elastic vertebra are the higher cervical segments. The lumbar segments, which bear more pressure, have higher Youngs Modulus than the cervical segments.

The foregoing tantalum, titanium, tantalum alloy, or titanium alloy all have a higher Youngs modulus than natural bone, although these metals are all significantly lower in Youngs modulus than many other metals including stainless steel. However, the design of the vertebral disc prosthesis of this invention may impart significant flexibility to the overall implant, so that the implant as a whole may have a much lower Youngs Modulus because of flexibility in the porous lattice structure of the instant design. Thus, in an embodiment, the vertebral disc prosthesis of this invention may be constructed with a different elastic modulus for different placements in the body.

In an embodiment, the lattices of the superior surface layer, the inferior surface layer and the midsection of the implant are roughened with a macro surface roughness. This may be accomplished with a technique such as grit blasting, acid etching, or plasma spray coating (also called thermal spray coating). In an embodiment, the lattices of the superior surface layer, the inferior surface layer and the midsection of the implant are coated with hydroxyapatite (HA) or tricalcium phosphate (TCP), with a coating thickness of approximately 25-35 μm. HA and TCP are well known as osteoconductive materials that encourage bone growth.

Thus, the bone-contacting spongy matrix may comprise four factors to encourage bone in-growth: the porosity of the spongy matrix layer that directly contacts the bone, the roughened surface, and a coating of HA or TCP. In addition, the hollow center 112 of the implants will encourage bone in-growth or through-growth.

In an embodiment, the vertebral disc prosthesis has a segment on one end adapted for connecting to a tool that can be used during surgery to assist in placing the implant in the appropriate location and positioning the implant as desired by the surgeon.

While the apparatuses of subject invention have been shown and described with reference to preferred embodiments, it is to be understood that any feature described in connection with one embodiment can be advantageously applied to other embodiments of the invention, even if not explicitly described in connection therewith, if such feature(s) are not mutually exclusive with other features of such embodiment. Nevertheless, those skilled in the art will readily appreciate that further changes or modifications may be made to devices and methods of the present invention without departing from the spirit and scope thereof.

What is claimed is:

1. A vertebral disc comprising:
a. a superior surface layer, an inferior surface layer, a midsection between the superior and inferior surface layers and a hollow center;
b. wherein the superior surface layer and inferior surface layer each comprise a layer of lattice with pore sizes in the range of 200-900 μm, with a porosity of each layer of 55-75%;
c. wherein the midsection of the implant comprises a random or pseudorandom lattice interposed between the superior surface layer and inferior surface layer with macro-pore sizes ranging from 0.5 mm to 5.0 mm; and
d. wherein there is a gradient of pore sizes along a longitudinal axis with the smallest pore sizes comprising the superior and inferior surfaces and the largest pore sizes comprising the midsection.

2. The vertebral disc of claim 1, wherein the lattices of superior surface layer and inferior surface layer each include a tantalum alloy or titanium alloy material of 0.5 mm to 2 mm thickness.

3. The vertebral disc of claim 1, wherein the lattice of the midsection includes a tantalum alloy or titanium alloy material with a thickness of between 4 mm and 14 mm.

4. The vertebral disc of claim 1, wherein the transition of pore sizes from the superior and inferior surface layers to the midsection is gradual.

5. The vertebral disc of claim 1, wherein the lattices of the superior surface layer, the inferior surface layer and the midsection are roughened with a macro surface roughness.

6. The vertebral disc of claim 1, where the lattices of the superior surface layer, the inferior surface layer and the midsection are coated with hydroxyapatite (HA) or tricalcium phosphate (TCP), with a coating thickness of approximately 25-35 um.

7. The vertebral disc of claim 1, wherein the superior surface layer and inferior surface layer each contact a vertebra.

8. The vertebral disc prosthesis of claim 7, wherein the superior surface layer and inferior surface layer each define a plane, wherein the superior surface plane and inferior surface plane are parallel.

9. The vertebral disc prosthesis of claim 7, wherein the superior surface layer and inferior surface layer each define a plane, and the superior surface plane and the inferior surface plane are non-parallel and at a 5° to 30° angle with respect to each other, such that a height at a first end of the midsection is less than a height of a second opposing end of the midsection.

10. A vertebral disc prosthesis for cervical, thoracic, lumbar disc arthrodesis, comprising:
a. a superior surface layer, an inferior surface layer, a midsection, and a hollow center on a longitudinal axis to serve as a graft window;
b. wherein the superior surface layer and inferior surface layer each comprises an approximately 0.5 to 2 mm thick layer of lattice made from tantalum alloy or titanium alloy material with pore sizes in the spongy matrix in the range of 200-900 μm, with a porosity of each layer of 55-75%;
c. wherein the midsection of the implant comprises a random or pseudorandom lattice interposed between the superior surface layer and inferior surface layer, wherein the lattice comprises tantalum alloy or titanium alloy material with a thickness of between 4 mm and 14 mm, and a pore size of 0.5 mm to 5.0 mm; and
d. wherein there is a gradient of pore sizes along the longitudinal axis with the smallest pore sizes comprising the superior and inferior surfaces and the largest pore sizes comprising the midsection.

11. The vertebral disc of claim 10, wherein the transition of thickness of the lattice and pore sizes from the superior and inferior surface layers to the midsection is gradual.

12. The vertebral disc of claim 10, wherein the lattices of the superior surface layer, inferior surface layer and midsection are roughened with a macro surface roughness of between about 200 μm and 900 μm.

13. The vertebral disc of claim 10, where the lattices of the superior surface layer, inferior surface layer and midsection are coated with hydroxyapatite (HA) or tricalcium phosphate (TCP), with a coating thickness of approximately 25-35 um.

14. A vertebral disc prosthesis for cervical, thoracic, lumbar disc arthrodesis, comprising:
a. a circular (torodial), kidney, or parallelepipedal shaped implant with a superior surface layer, an inferior surface layer, a midsection, and a hollow center on a longitudinal axis to serve as a graft window;
b. wherein each surface layer contacts a vertebra;
c. wherein the superior surface layer and inferior surface layer each comprises an approximately 0.5 mm to 2 mm thick layer of lattice made from tantalum alloy or titanium alloy material with pore sizes in the spongy matrix in the range of 200-900 μm, with a porosity of each layer of 55-75%;
d. wherein the midsection of the implant comprises a random or pseudorandom lattice interposed between the superior surface layer and inferior surface layer, wherein the lattice comprises tantalum alloy or titanium alloy material with a thickness of between 4 mm and 14 mm, and a pore size of 0.5 mm to 5.0 mm;
e. wherein the lattices of the superior surface layer, inferior surface layer and midsection are roughened with a macro surface roughness; and
f. where the lattices of the superior surface layer, inferior surface layer and midsection are coated with hydroxyapatite (HA) or tricalcium phosphate (TCP), with a coating thickness of approximately 25-35 um.

15. The vertebral disc prosthesis of claim 14, wherein there is a gradient of pore sizes along the longitudinal axis with the smallest pore sizes at the superior and inferior surfaces and the largest pore sizes in the midsection.

16. The vertebral disc prosthesis of claim 14, wherein the superior surface layer and inferior surface layer each define a plane, wherein the superior surface plane and inferior surface plane are parallel.

17. The vertebral disc prosthesis of claim 14, wherein the superior surface layer and inferior surface layer each define a plane, and the superior surface plane and inferior surface plane are non-parallel and at a 5° to 30° angle with respect to each other, such that a height at a first end of the midsection is less than a height of a second opposing end of the midsection.

18. The vertebral disc of claim 14, wherein the prosthesis is made by additive manufacturing.
